(19) United States
(12) Patent Application Publication
(10) Pub. No.: US 2010/0125289 A1
(43) Pub. Date: May 20, 2010

(54) SCALPEL BLADE HAVING DUAL INDENTATIONS ON BACK EDGE

(75) Inventor: Rajiv Kehr, Gurgaon (IN)

Correspondence Address: CLIFFORD L TAGER, Box 467518, Atlanta, GA, 31146, US

(73) Assignee: Aditya Dispomed Products Private Ltd., Gurgaon (IN)

(21) Appl. No.: 12/272,928

(22) Filed: Nov. 18, 2008

(51) Int. Cl. A61B 17/32

(52) U.S. Cl. 606/167

(57) ABSTRACT

The present invention is directed to a scalpel blade having a longitudinal axis, the scalpel blade preferably comprising a first edge coincident with the longitudinal axis, a second edge coincident with the longitudinal axis, a cutting edge located along at least a portion of the first edge, and a first indentation in juxtaposition with and spanning at least a portion of the second edge, wherein the first indentation is offset in a first direction from the second edge. The scalpel blade preferably further comprises a second indentation in juxtaposition with and spanning at least a portion of the first indentation, wherein the second indentation is offset in a direction opposite from the second edge. The scalpel blade can either be removably attached to a reusable scalpel handle or indelibly attached to a disposable scalpel.

### BACKGROUND OF THE INVENTION

1. Technical Field

The present invention is directed to blades. More particularly, the present invention is directed to scalpel blades. The present invention finds particular utility in the medical field, as well as in applications that require a strong blade, that is, a blade able to withstand a large maximum load before failure.

2. Background Information

Various types of blades are known in the art. For example, in the field of arts and crafts, fine edged blades are used for cutting and shaping materials such as wood, balsa, cardboard, plastics, Bristol board, and the like. The use of scalpel blades in various applications is also well known.

As understood by one skilled in the art, a scalpel comprises a scalpel handle and a scalpel blade. A scalpel blade is a relatively small and extremely sharp instrument used for surgery and anatomical dissections, as well as for various arts and crafts. Historically, a scalpel was comprised of a single piece of metal having a blade portion and a handle portion. The blade portion was subject to repeated sharpening in order for the scalpel to be reused.

In or about 1915, a system of a disposable scalpel blade attachable to a reusable metal handle was developed. Specifically, the scalpel handle had a blade platform with a longitudinal protrusion, and the scalpel blade had a longitudinal hole that corresponded to the dimensions of the handle's protrusion. The scalpel blade fit over the protrusion and was able to be removably secured to the handle.

Over time, different manufacturers used different fitment dimensions for the longitudinal hole in the scalpel blade and the protrusion on the handle, which made compatibility an issue.

In an effort to bring uniformity to the fitment dimensions for detachable scalpel blades and the handles with which they are used, the fitment dimensions were harmonized worldwide in 1985 by the International Organization for Standardization (ISO).

The ISO memorialized the fitment standards as ISO 7740:1985—Instruments for surgery—Scalpels with detachable blades—Fitting dimensions, which standards have been adopted worldwide. ISO 7740 sets forth two fitment standards, one for blades and handles with a small fitting feature (fitment 3), the other for blades and handles with a large fitting feature (fitment 4). As part of this standard, the minimum and maximum dimensions of the scalpel blade thickness (for the fitting area) were also established (i.e., 0.37-0.42 millimeters).

Scalpel blades are typically made of either high carbon steel or stainless steel. Scalpel blades are by necessity relatively very hard, since hardness is a necessity for a sharp cutting edge. Unfortunately, the greater the hardness of the blade, the more brittle the blade, which poses severe hazards if a blade breaks in vivo during surgery.

Although one option to make scalpel blades stronger would be to make the blades uniformly thicker, the ISO 7740 standard dictates the maximum blade thickness.

Accordingly, there is need for a strong scalpel blade that conforms to the ISO 7740 standards.

### SUMMARY OF THE INVENTION

In a first embodiment of a scalpel blade of the present invention having a longitudinal axis, the scalpel blade preferably comprises a first edge coincident with the longitudinal axis, a second edge coincident with the longitudinal axis, a cutting edge located along at least a portion of the first edge, and a first indentation in juxtaposition with and spanning at least a portion of the second edge, wherein the first indentation is offset in a first direction from the second edge.

In the first embodiment, the scalpel blade preferably further comprises a second indentation in juxtaposition with and spanning at least a portion of the first indentation, wherein the second indentation is offset in a second direction from the second edge. The first direction is opposite the second direction, and the blade can either be removably or indelibly attached to a handle.

In a second embodiment of a scalpel blade of the present invention having a longitudinal axis, a first edge and a second edge coincident with the longitudinal axis and a cutting edge located along at least a portion of the first edge, the scalpel blade preferably comprises a first indentation spanning at least a portion of the second edge.

In the second embodiment, the scalpel blade preferably further comprises a second indentation spanning at least a portion of the first indentation. The first indentation is offset in a first direction from the second edge, and the second indentation is offset in a direction opposite the first direction. The first indentation is located in juxtaposition with at least a portion of the second edge, and the second indentation is located in juxtaposition with at least a portion of the first indentation.

In a third embodiment of a scalpel blade of the present invention having a longitudinal axis, the scalpel blade preferably comprises a first edge and a second edge coincident with the longitudinal axis, a cutting edge located along at least a portion of the first edge, and a first indentation in juxtaposition with at least a portion of the second edge, the first indentation being offset in a first direction.

In the third embodiment, the scalpel blade preferably further comprises a second indentation in juxtaposition with at least a portion of the first indentation, the second indentation being offset in a second direction. The first direction is opposite the second direction. The first indentation spans at least a portion of the second edge, and the second indentation spans at least a portion of the first indentation.

A fourth embodiment of the present invention is a cutting instrument having a longitudinal axis and preferably comprises a first edge and a second edge coincident with the longitudinal axis, a cutting edge located along at least a portion of the first edge, and a first indentation spanning at least a portion of the cutting edge, the first indentation being offset in a first direction.

In the fourth embodiment, the cutting instrument preferably further comprises a second indentation spanning at least a portion of the first indentation, the second indentation being offset in a direction opposite the first direction. The second indentation is located in juxtaposition with at least a portion of the first indentation, and the first indentation is located in juxtaposition with at least a portion of the second edge.

A method of fabricating a scalpel blade of the present invention from a piece of metal preferably comprises the steps of stamping out a blank from the metal, the blank comprising an outline of the scalpel blade having a first edge and a second edge, stamping out an aperture from the blank, forming a first indentation spanning at least a portion of the second edge, the first indentation being offset in a first direction, and creating a cutting edge located along at least a portion of the first edge.

The method of fabricating a scalpel blade of the present invention preferably further comprises the step of forming a second indentation spanning at least a portion of the first indentation, the second indentation being offset in a second direction.

In the step of forming the second indentation, the second direction is opposite the first direction. In the step of forming the first indentation, the first indentation is located in juxtaposition with at least a portion of the second edge, and, in the step of forming the second indentation, the second indentation is located in juxtaposition with at least a portion of the first indentation.

The method of fabricating a scalpel blade of the present invention preferably further comprises the step of heat treating the blank prior to the step of creating the cutting edge, and the step of attaching the fabricated scalpel blade to a handle.

### BRIEF DESCRIPTION OF THE DRAWINGS

FIG. 1 illustrates a profile view of a first embodiment of the present invention, specifically, a number 10 scalpel blade.

FIG. 2 is a cross-sectional view of the scalpel blade shown in FIG. 1.

FIG. 3 illustrates a profile view of a second embodiment of the present invention, specifically, a number 11 scalpel blade.

FIG. 4 is a cross-sectional view of the scalpel blade shown in FIG. 3.

FIG. 5 illustrates a profile view of a third embodiment of the present invention, specifically, a number 12 scalpel blade.

FIG. 6 is a cross-sectional view of the scalpel blade shown in FIG. 5.

FIG. 7 illustrates a profile view of a fourth embodiment of the present invention, specifically, a number 15 scalpel blade.

FIG. 8 is a cross-sectional view of the scalpel blade shown in FIG. 7.

FIG. 9 illustrates a profile view of a fifth embodiment of the present invention, specifically, a number 20 scalpel blade.

FIG. 10 is a cross-sectional view of the scalpel blade shown in FIG. 9.

FIG. 11 illustrates a profile view of a sixth embodiment of the present invention, specifically, a number 21 scalpel blade.

FIG. 12 is a cross-sectional view of the scalpel blade shown in FIG. 11.

FIG. 13 illustrates a profile view of a seventh embodiment of the present invention, specifically, a number 22 scalpel blade.

FIG. 14 is a cross-sectional view of the scalpel blade shown in FIG. 13.

FIG. 15 illustrates a profile view of an eighth embodiment of the present invention, specifically, a number 23 scalpel blade.

FIG. 16 is a cross-sectional view of the scalpel blade shown in FIG. 15.

FIG. 17 illustrates a profile view of a ninth embodiment of the present invention, specifically, a number 24 scalpel blade.

FIG. 18 is a cross-sectional view of the scalpel blade shown in FIG. 17.

FIG. 19 illustrates a profile view of a conventional scalpel handle.

FIG. 20 illustrates a profile view of a disposable scalpel comprising a conventional handle and a scalpel blade of the present invention.

FIG. 21 illustrates a side view of a preferred embodiment of a top half of a forming die used in the fabrication process of the scalpel blades of the present invention.

FIG. 22 is a detailed view of a portion of the forming die shown in FIG. 21.

### DESCRIPTION OF THE PREFERRED EMBODIMENT(S)

Scalpel blades are available in various sizes, and each size is given a specific blade number, such as number 9, 10, 10A, 11, 11P, 12, 12B, 12D, 13, 14, 15, 15A, 15B, 15C, 15T, 16, 17, 18, 19, 20, 21, 22, 22A, 23, 24, 24D, 25, 25A, 26, 27, 34, 36, 36D, 60, PM40, PM40B, PM60, and PM60B. The most common scalpel blade sizes used in the medical industry are numbers 10, 11, 12, 15, 20, 21, 22, 23 and 24.

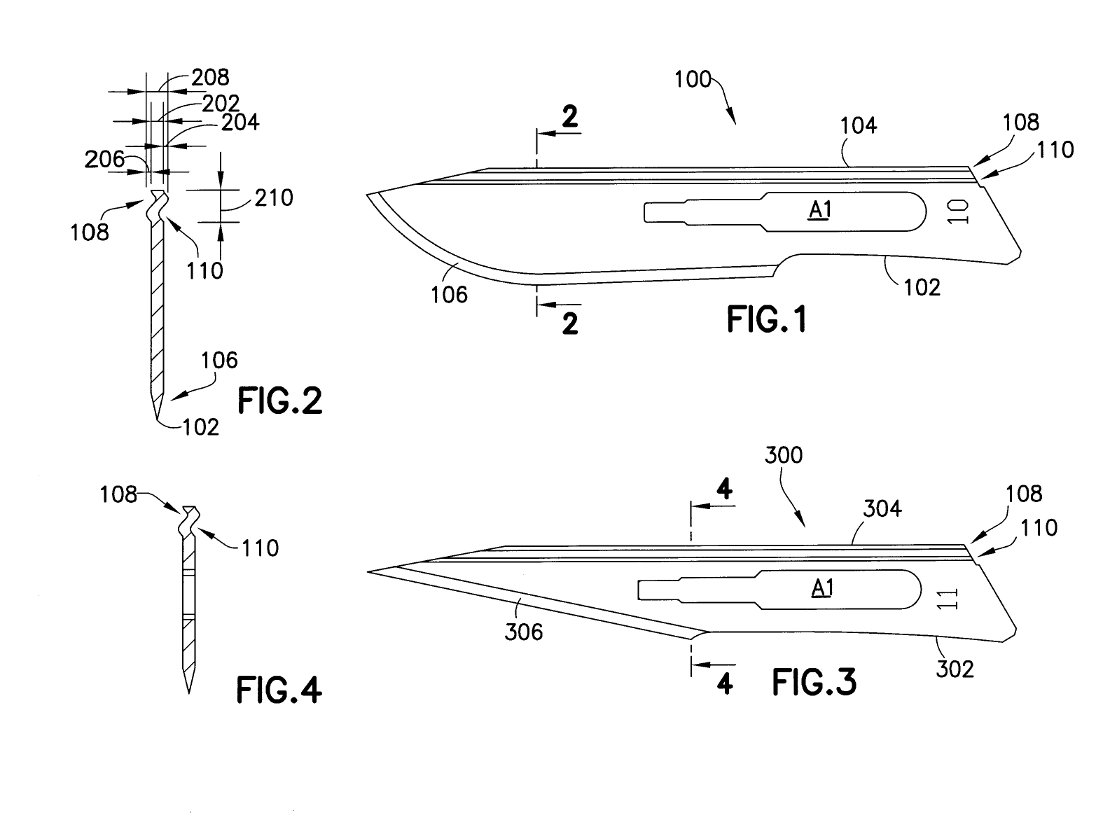

While FIGS. 1 through 18 illustrate various preferred embodiments of the present invention with respect to the most common scalpel blade sizes, the present invention is equally applicable to all scalpel blade sizes, as will be apparent to one skilled in the art.

FIG. 1 is a profile view of a first embodiment of the present invention, specifically, a number 10 scalpel blade. FIG. 2 is a cross-sectional view of the scalpel blade shown in FIG. 1.

As shown in FIG. 1, scalpel blade 100 preferably comprises first edge 102, second edge 104, cutting edge 106 located along a portion of first edge 102, first indentation 108 and second indentation 110. Second edge 104 is commonly referred to in the art as the back edge.

FIG. 3 illustrates a profile view of a second embodiment of the present invention, specifically, a number 11 scalpel blade. As shown in FIG. 3, scalpel blade 300 preferably comprises first edge 302, second edge 304, cutting edge 306 located along a portion of first edge 302, first indentation 108 and second indentation 110. FIG. 4 is a cross-sectional view of the scalpel blade shown in FIG. 3.

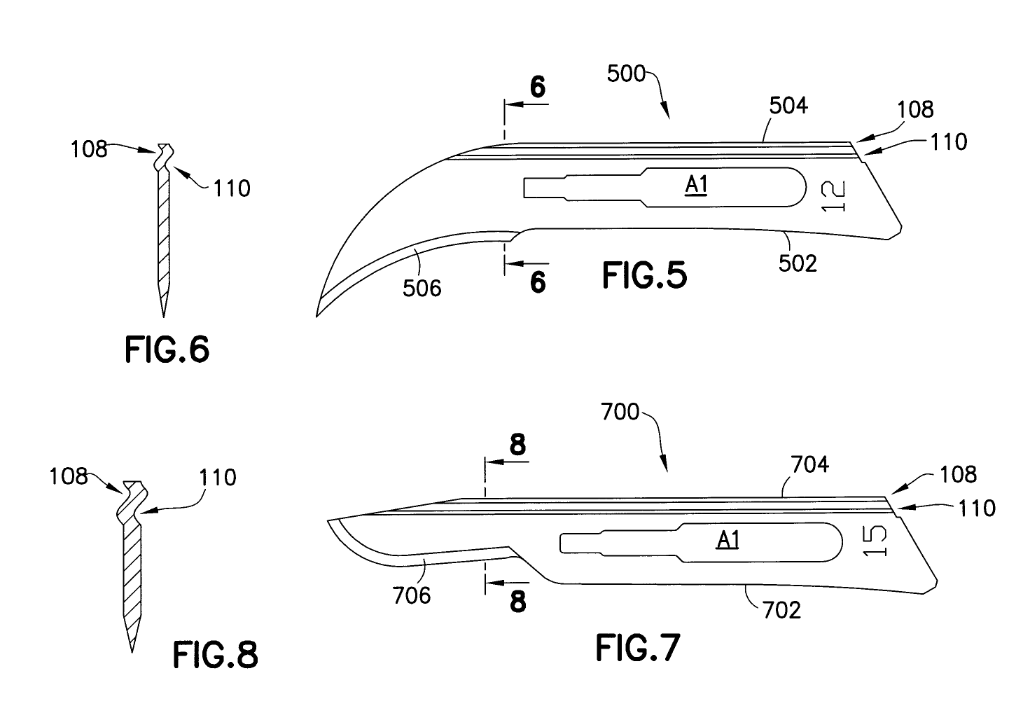

FIG. 5 illustrates a profile view of a third embodiment of the present invention, specifically, a number 12 scalpel blade. As shown in FIG. 5, scalpel blade 500 preferably comprises first edge 502, second edge 504, cutting edge 506 located along a portion of first edge 502, first indentation 108 and second indentation 110. FIG. 6 is a cross-sectional view of the scalpel blade shown in FIG. 5.

FIG. 7 illustrates a profile view of a fourth embodiment of the present invention, specifically, a number 15 scalpel blade. As shown in FIG. 7, scalpel blade 700 preferably comprises first edge 702, second edge 704, cutting edge 706 located along a portion of first edge 702, first indentation 108 and second indentation 110. FIG. 8 is a cross-sectional view of the scalpel blade shown in FIG. 7.

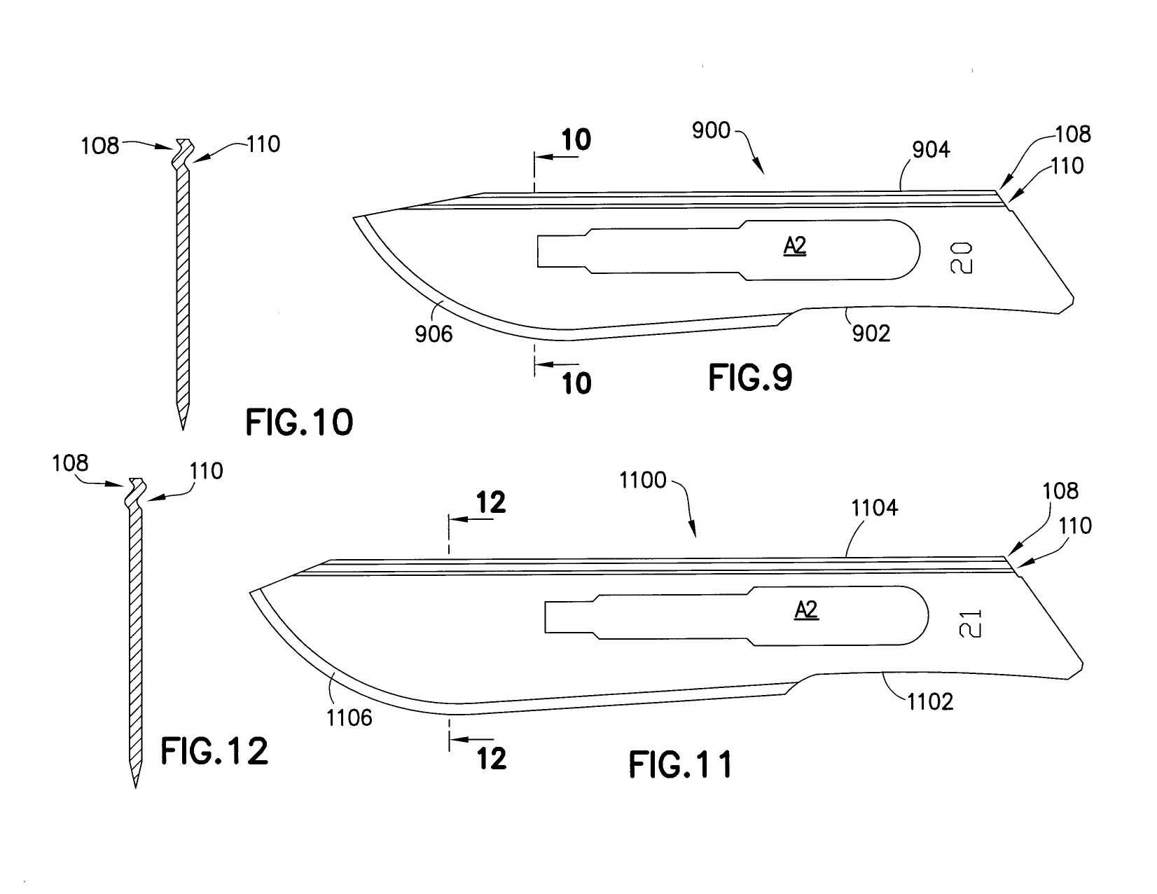

FIG. 9 illustrates a profile view of a fifth embodiment of the present invention, specifically, a number 20 scalpel blade. As shown in FIG. 9, scalpel blade 900 preferably comprises first edge 902, second edge 904, cutting edge 906 located along a portion of first edge 902, first indentation 108 and second indentation 110. FIG. 10 is a cross-sectional view of the scalpel blade shown in FIG. 9.

FIG. 11 illustrates a profile view of a sixth embodiment of the present invention, specifically, a number 21 scalpel blade. As shown in FIG. 11, scalpel blade 1100 preferably comprises first edge 1102, second edge 1104, cutting edge 1106 located along a portion of first edge 1102, first indentation 108 and second indentation 110. FIG. 12 is a cross-sectional view of the scalpel blade shown in FIG. 11.

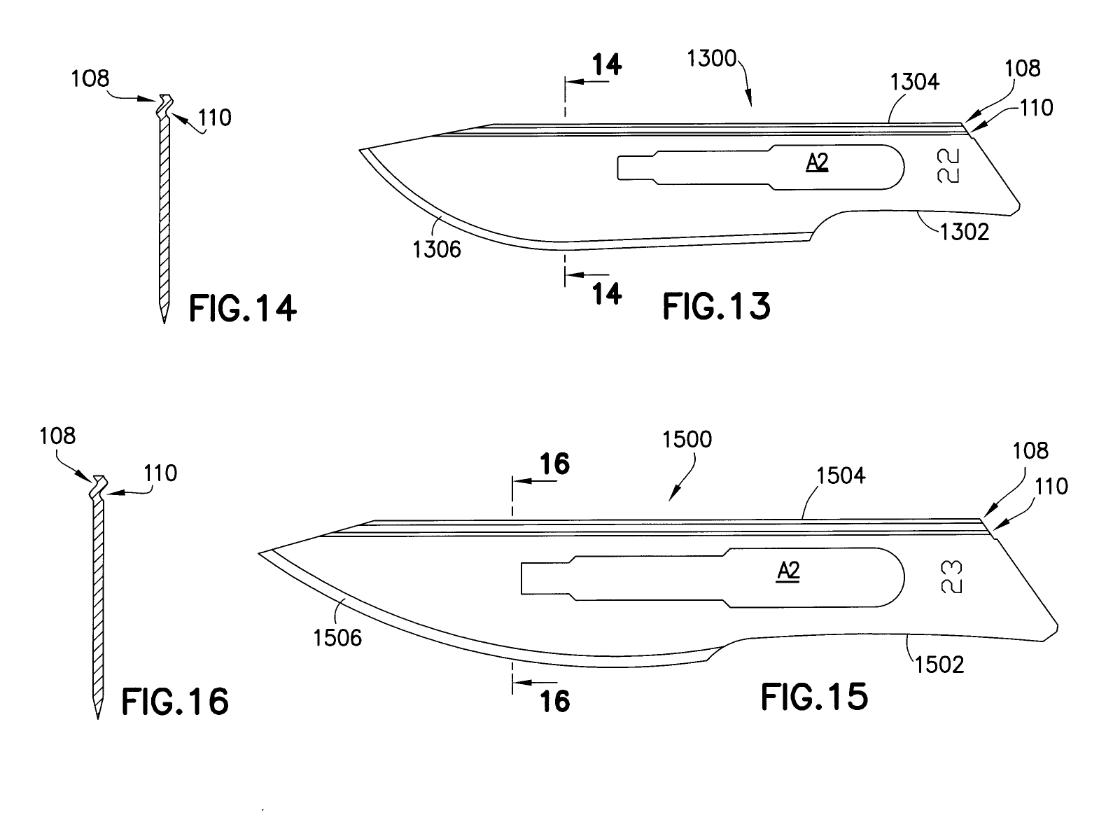

FIG. 13 illustrates a profile view of a seventh embodiment of the present invention, specifically, a number 22 scalpel blade. As shown in FIG. 13, scalpel blade 1300 preferably comprises first edge 1302, second edge 1304, cutting edge 1306 located along a portion of first edge 1302, first indentation 108 and second indentation 110. FIG. 14 is a cross-sectional view of the scalpel blade shown in FIG. 13.

FIG. 15 illustrates a profile view of an eighth embodiment of the present invention, specifically, a number 23 scalpel blade. As shown in FIG. 15, scalpel blade 1500 preferably comprises first edge 1502, second edge 1504, cutting edge 1506 located along a portion of first edge 1502, first indentation 108 and second indentation 110. FIG. 16 is a cross-sectional view of the scalpel blade shown in FIG. 15.

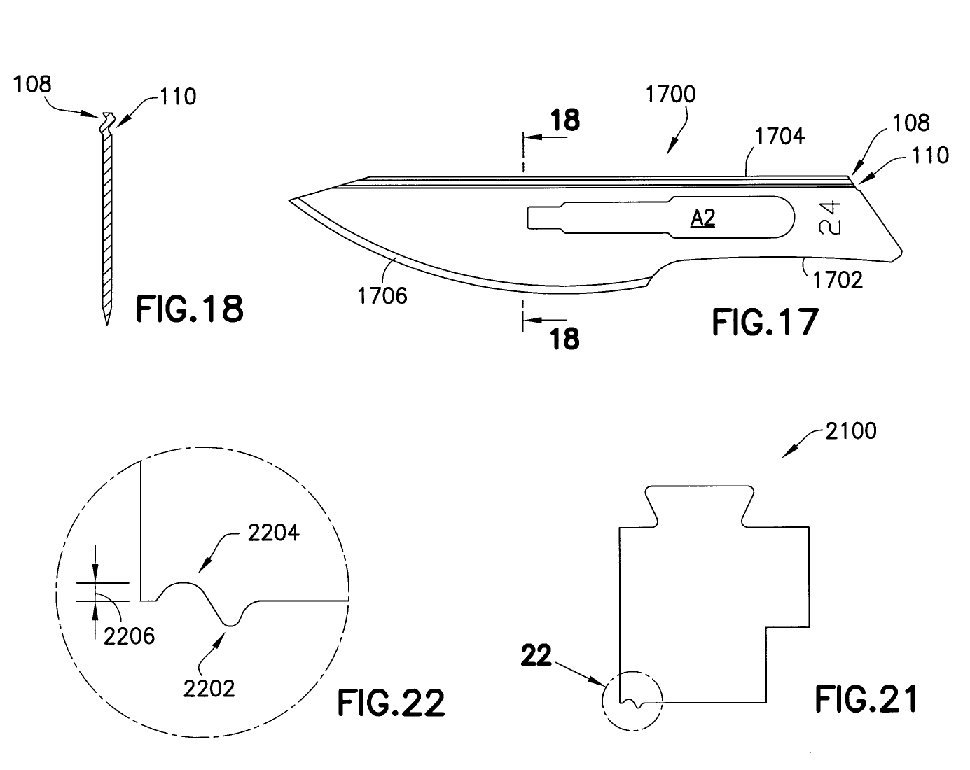

FIG. 17 illustrates a profile view of a ninth embodiment of the present invention, specifically, a number 24 scalpel blade. As shown in FIG. 17, scalpel blade 1700 preferably comprises first edge 1702, second edge 1704, cutting edge 1706 located along a portion of first edge 1702, first indentation 108 and second indentation 110. FIG. 18 is a cross-sectional view of the scalpel blade shown in FIG. 17.

As known in the art, the fitment dimensions for the apertures of scalpel blade numbers 9 through 17 are in accordance with ISO 7740 for fitment 3, and the fitment dimensions for the apertures of scalpel blade numbers 18 and above are in accordance with ISO 7740 for fitment 4.

Accordingly, with reference to FIGS. 1, 3, 5 and 7, the fitment dimensions for aperture Al preferably conform to ISO 7740 for fitment 3, and with reference to FIGS. 9, 11, 13, 15 and 17, the fitment dimensions for aperture A2 preferably conform to ISO 7740 for fitment 4. Apertures A1 and/or A2 may alternatively have other fitment dimensions that do not conform to ISO 7740, as will be apparent to one skilled in the art.

In the preferred embodiment, and with reference to FIGS. 1 through 18, first indentation 108 is located in close proximity to the second edge of the scalpel blade, and second indentation 110 is located in close proximity to first indentation 108. Alternatively, first indentation 108 could be located either closer to or further away from the second edge, and second indentation 110 could be located either closer to or further away from first indentation 108, as will be apparent to one skilled in the art.

First indentation 108 is preferably offset in a first direction from the second edge, and second indentation 110 is preferably offset in a direction opposite the first direction. Alternatively, first indentation 108 could be offset in the direction of second indentation 110 and second indentation 110 could be offset in the direction of first indentation 108, or both the first and second indentations could be offset in the same direction, as will be apparent to one skilled in the art.

First indentation 108 and second indentation 110 preferably span the entire length of the second edge and continue their respective spans across the relevant horizon of the scalpel blade, as shown in FIGS. 1 through 18. Alternatively, the span of the first and/or the second indentation can be less than the entire length of the second edge, e.g., having a span corresponding to the horizontal component of the cutting edge (e.g., 106, FIG. 1), and/or the span of the first indentation could be greater than, less than or equal to the span of the second indentation. Other span dimensions for the first and/or the second indentation, relative either to the second edge or to each other, will be apparent to one skilled in the art.

In the preferred embodiment, scalpel blades of the present invention comprise two indentations (i.e., 108 and 110), one being relatively symmetrical to the other in terms of height (i.e., the distance between the upper and lower edges of the indentation), depth and shape (i.e., relatively “u”-shaped). Alternatively, the scalpel blade may comprise more or less than two indentations, the shape of one or both indentations may be different than shown, and the first indentation need not be relatively symmetrical to the second indentation, as will be apparent to one skilled in the art.

As shown in FIG. 2, width 202 of scalpel blade 100, not including the protrusions created by first and second indentations 108 and 110, respectively, is preferably 0.395±0.025 mm, which dimensions correspond to the minimum and maximum dimensions set forth in ISO 7740 (i.e., between 0.37 mm and 0.42 mm).

First indentation 108 preferably causes that portion of scalpel blade 100 that comprises first indentation 108 to protrude a distance, shown by reference 204, of about 0.150 mm, relative to width 202. Similarly, second indentation 110 preferably causes that portion of scalpel blade 100 that comprises second indentation 110 to protrude a distance, shown by reference 206, of about 0.150 mm, relative to width 202. Overall width 208 of scalpel blade 100, including the protrusions created by first and second indentations 108 and 110, respectively, is therefore preferably about 0.695±0.025 mm.

The indentation area, i.e., the distance between the upper-most edge of first indentation 108 and the lower-most edge of second indentation 110, shown by reference 210, is preferable about 1.0 mm.

In the preferred embodiment, the dimensions discussed above with reference to FIGS. 1 and 2, i.e., reference numbers 202 through 210, are identical for all other scalpel blades of the present invention shown in FIGS. 3 through 18. Other dimensions for width 202, protrusion widths 204 and 206, overall width 208 and indentation area 210 for each individual scalpel blade will be apparent to one skilled in the art.

The scalpel blades of the present invention shown in FIGS. 1 through 18 find particular utility as replacement scalpel blades that are attachable to and usable with conventional reusable scalpel handles, as well as scalpel blades affixed to and made a part of disposable scalpels.

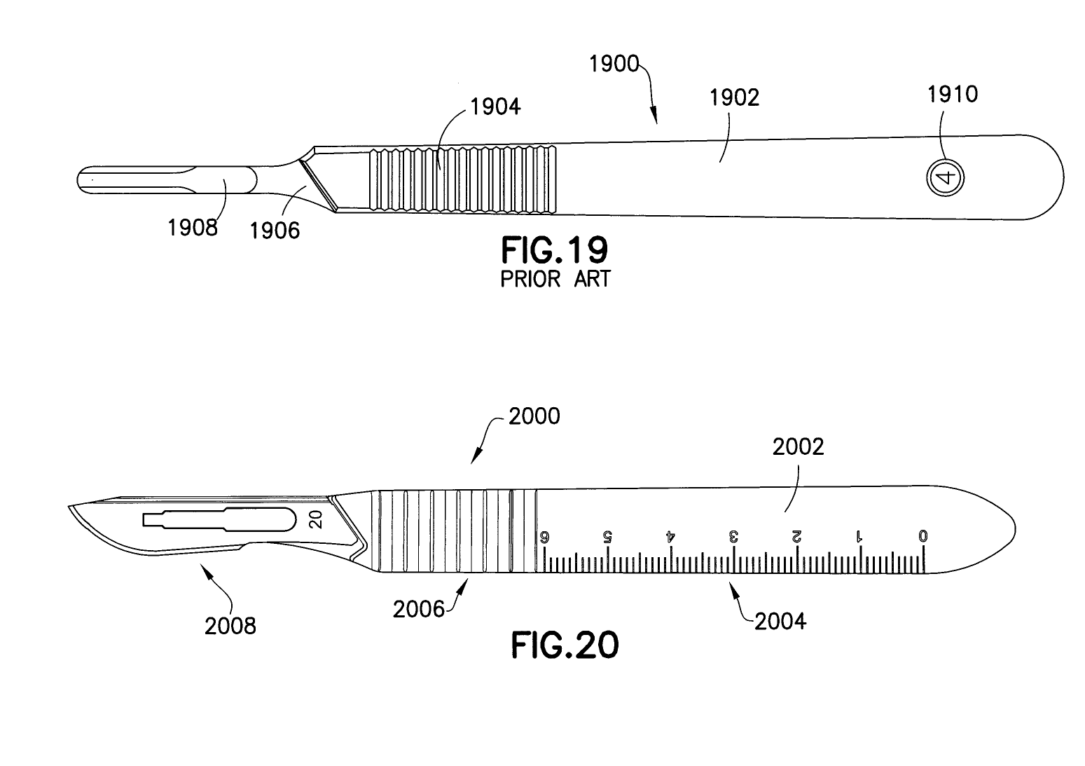

Turning now to FIG. 19, a profile view of a conventional reusable scalpel handle is shown. Conventional reusable handle 1900 typically comprises handle portion 1902 having non-slip grip area 1904, blade platform 1906 and raised projection 1908. Conventional reusable scalpel handles are typically fabricated from a material that can be sanitized, i.e., autoclaved, as may be required from time to time by applicable heath codes and standards.

As will be appreciated by one skilled in the art, the dimensions of blade platform 1906 and raised projection 1908 are specified by ISO 7740 for both fitment 3 and fitment 4. Conventional reusable handles typically also include a graphic, such as shown at reference 1910, which indicates whether the handle is compatible with fitment 3 or fitment 4 scalpel blades.

The scalpel blades shown with reference to FIGS. 1 through 8 are preferably removably attachable to a handle compatible with fitment 3 dimensions, while the scalpel blades shown with reference to FIGS. 9 through 18 are preferably removably attachable onto a handle compatible with fitment 4 dimensions.

Turning now to FIG. 20, a profile view of a disposable scalpel comprising a conventional handle and a scalpel blade of the present invention is shown. Disposable scalpel 2000 preferably comprises conventional handle 2002 having ruler markings 2004 and non-slip grip area 2006, as well as scalpel blade 2008 of the present invention, shown as blade number 20 , rigidly and indelibly affixed thereto in a conventional manner.

As known in the art, the scalpel blade affixed to a disposable scalpel, such as scalpel blade 2008, is not designed to be removable or replaceable. In medical applications, scalpel blades are used only during a single operation or procedure, even if just for a single cut, to prevent cross-contamination. Since a disposable scalpel is designed to be discarded after use, conventional disposable scalpel handles are typically fabricated from an inexpensive material, such as a polycarbonate, acrylonitrile butadiene styrene (ABS) or other plastic material.

As will be appreciated by one skilled in the art, any of the scalpel blades shown with reference to FIGS. 1 through 18, as well as any other scalpel blade incorporating the first and/or a second indentation of the present invention, are able to be removably attached to and used with any applicable reusable scalpel handle, as well as incorporated into and made a part of any applicable disposable scalpel.

Scalpel blades made in accordance with the present invention are preferably manufactured either from high carbon steel or stainless steel. Specifically, the high carbon steel preferably conforms to British Standard BS 2982:1992, commonly referred to as 125Cr1, having the following constituent components, to wit: carbon, 1.20 to 1.30%; silicon, 0.10 to 0.35%; manganese, 0.10 to 0.45%; chromium, 0.10 to 0.40%; sulfur, maximum of 0.025%; and phosphorus, maximum of 0.035%. The stainless steel preferably conforms to British Standard BS 5194:1992, commonly referred to as grade 13C26 by Sandvik, Sweden, having the following constituent components, to wit: carbon, 0.6 to 0.7%; silicon, maximum of 0.5%; manganese, maximum of 1.0%; chromium, 12.0 to 13.5%; sulfur, maximum of 0.025%; phosphorus, maximum of 0.03%; and nickel, maximum of 0.5%. Other grades of high carbon steel and stainless steel, as well as other scalpel blade materials, will be apparent to one skilled in the art.

The scalpel blades of the present invention are preferably manufactured by feeding a coil of material, e.g., high carbon steel or stainless steel conforming to the above specifications and having the requisite material thickness (i.e., width 202, FIG. 2), into a conventional press having a progressive tool.

As will be appreciated by one skilled in the art, as the material is fed through the press, the press preferably stamps out a general outline of the scalpel blade (also referred to as a “blank”), stamps out the requisite aperture (i.e., A1 (fitment 3) or A2 (fitment 4), depending on the blade number being manufactured), and stamps the blade number and any other requisite or desired indicia (such as a logo and the word “stainless”, where applicable) onto the blade in the conventional locations and in a conventional manner. The press also preferably forms the first and second indentations (108 and 110, respectively, FIGS. 1-18) onto the blade, as discussed in more detail with reference to FIGS. 21 and 22 below.

Thereafter, the blade is preferably heat treated to harden the blade and polished in a conventional manner, then inspected for quality control purposes (e.g., surface defects, aperture fitment dimensions, hardness and/or deflection characteristics). Upon passing quality control, the requisite portion of the first edge of the blade (e.g., 102, FIG. 1) is preferably ground, buffed and honed in a conventional manner to create the cutting edge of the blade (e.g., 106, FIG. 1). The sharpened scalpel blade is cleaned, preferably ultrasonically, to remove any grease, metal particles and other debris remaining thereon, and then preferably inspected visually for grinding, buffing, cleaning and surface defects before being packaged and sterilized in a conventional manner.

In the preferred embodiment, the cutting edge comprises a single facet on each side of the scalpel blade, and the angle of each facet of the cutting edge is about 13 degrees ±1 degree. Alternatively, the cutting edge could comprise two facets on each side of the scalpel blade, such as found on conventional razor blades, as will be appreciated by one skilled in the art.

In the preferred embodiment, the first and second indentations (108 and 110, respectively, FIGS. 1-18) are formed using a two-part forming die comprising a top half and a bottom half.

FIG. 21 illustrates a side view of a preferred embodiment of the top half of the forming die used in the fabrication process of the scalpel blades of the present invention. FIG. 22 is a detailed view of a portion of the forming die shown in FIG. 21.

As shown in FIGS. 21 and 22, top half of forming die 2100 preferably comprises projection 2202 and depression 2204. The bottom half of the forming die (not shown) is preferably symmetrical to top half 2100 such that projection 2202 is vertically aligned with and located over a depression on the bottom half of the forming die, and depression 2204 is vertically aligned with and located over a projection on the bottom half of the forming die.

In use, the scalpel blank is preferably located between the top and bottom halves of the forming die such that projection 2202 is positioned just below the second edge (e.g., 104, FIG. 1) thereof. When the top half of the forming die is pressed towards the bottom half of the forming die, projection 2202 on the top half and its corresponding depression on the bottom half form first indentation 108, and depression 2204 on the top half and its corresponding projection on the bottom half form second indentation 110.

As will be appreciated by one skilled in the art, top half of forming die 2100 preferably comprises a configuration, such as the dual-tenon dovetail shown in FIG. 21 located on the top edge thereof, so that it can be affixed to and easily removed from the press, thereby facilitating the periodic repair and eventual replacement thereof. The corresponding bottom half of the forming die is preferably a plate which sits in a die cavity of the press.

In the preferred embodiment, the depth of depression 2204, relative to the bottom edge of the forming die, as shown by reference 2206, is 0.180 millimeters. As discussed above, first indentation 108 is preferably substantially symmetrical to second indentation 110 in terms of height, depth and shape. Thus, the dimensions of depression 2204, as well as the depression on the bottom half of the forming die which is vertically aligned with and located under projection 2202, are preferably substantially identical. Similarly, the dimensions of projection 2202, as well as the projection on the bottom half of the forming die which is vertically aligned with and located under depression 2204, are preferably substantially identical.

The overall length of the forming die preferably takes into account the longest scalpel blade to be fabricated in accordance with the present invention. In the preferred embodiment, the overall length of the forming die is 40 millimeters and the overall width is 9 millimeters.

Other methods of manufacture, cutting edge angles and configurations, forming die projection and depression configurations, spacing, shape, dimensions (whether for producing symmetrical or non-symmetrical first and second indentations 108 and 110, respectively) and the direction of the first indentation relative to the second indentation, will be apparent to one skilled in the art.

Scalpel blades of the present invention, specifically, scalpel blades 100 (i.e., blade number 10 ), 300 (i.e., blade number 11 ) and 700 (i.e., blade number 15 ), were manufactured and tested to determine the maximum load that could be applied thereto before the blades failed (i.e., ruptured), as well as the blade's deflection at maximum load.

These tests were also conducted on the same sized scalpel blades manufactured by Bard-Parker and marketed under the Rib-Back™ mark, which blades comprise a solid longitudinal rib located along the scalpel blade's back edge. The longitudinal rib is wider than the remaining portion of the blade and overhangs the blade on both sides of the back edge. The Rib-Back™ scalpel blade is believed to be substantially described in Great Britain Patent GB 433,259 to Bard-Parker Company, Inc. (now a division of Becton, Dickinson and Company), and is considered in the industry to be a premium blade in terms of strength.

The testing was conducted on a Lloyd Instrument LF Plus universal testing machine suitable for tensile and compressive testing of loads up to 1000 Newtons. The machine uses high accuracy interchangeable XLC Series load cells. The load measuring system exceeds the requirements of all recognized international standards, and has a data sampling rate of 8 kHz.

A test fixture was designed to hold each scalpel blade in a specific location, and the tests were conducted using MIL STD GGH80 as a guideline. Specifically, each blade was clamped such that a bit more than the tang end of the blade and the aperture were secured by the clamp, while the remainder of the cutting edge protruded therefrom and extended outward in a horizontal position. The load was positioned 6 millimeters from the left edge of the aperture, and the load was applied in a downward direction with the load applicator mounted directly below the load cell.

The test protocol was controlled by the instrument's computer using NEXYGEN software supplied by the instrument's manufacturer, which lowered the load cell onto the blade until a preset load was applied thereto. The NEXYGEN software complies with the requirements of the Food and Drug Administration (FDA), 21 CFR Part II, ensuring the integrity of the data since a user is not able to modify the data.

Each blade was preloaded with a compressive force of 0.500 N. Thereafter, the load was applied at a speed of 5.00 mm/min until the blade failed. The instrument measured, graphed and recorded the maximum load to failure in Newtons (N), and the deflection of the blade at maximum load in millimeters (mm).

Testing was performed on the same day by the same test operator on numbers 10, 11 and 15 carbon steel scalpel blades of the present invention and those manufactured by Bard-Parker and marketed under the Rib-Back™ mark. The batch or lot numbers from the boxes of Bard-Parker scalpel blades tested are as follows: 7064063 (number 10 blades); 7183484 (number 11 blades); and 7156286 (number 15 blades).

Fifteen (15) Bard-Parker blade samples of each blade size were tested, while for the present invention, fifteen (15) samples of number 10 blades, nineteen (19) samples of number 11 blades, and six (6) samples of number 15 blades were tested. The data for the maximum load to failure (Max Load) and the deflection of the blade at maximum load (Def@ Max) were averaged for each blade size by blade manufacturer and are summarized below in Table 1.

TABLE 1

| | Present Invention | | Bard-Parker Rib-Back ™ | |
|---|---|---|---|---|
| Blade Size | Max Load | Def @ Max | Max Load | Def @ Max |
| 10 | 84.64 N | 2.26 mm | 87.95 N | 2.67 mm |
| 11 | 70.26 N | 2.67 mm | 67.36 N | 2.30 mm |
| 15 | 72.68 N | 2.85 mm | 63.18 N | 2.40 mm |

As will be appreciated by one skilled in the art, the number 10 blades were the strongest of the three blade sizes, which would be expected given the larger cross-sectional area of the number 10 blade relative to the number 11 and number 15 blades.

As can be seen by the test results summarized in Table 1, the scalpel blades of the present invention compare favorably with the Bard-Parker Rib-Back™ scalpel blades.

Although illustrative embodiments of the present invention have been described in detail with reference to the accompanying drawings, it is to be understood that the invention is not limited to those precise embodiments. Various changes or modifications may be effected therein by one skilled in the art without departing from the scope or spirit of the invention.

What I claim as my invention is:

1. A scalpel blade having a longitudinal axis, said scalpel blade comprising:
a first edge coincident with the longitudinal axis;
a second edge coincident with the longitudinal axis;
a cutting edge located along at least a portion of the first edge; and
a first indentation in juxtaposition with and spanning at least a portion of the second edge, said first indentation being offset in a first direction from the second edge.

2. The scalpel blade of claim 1, said scalpel blade further comprising a second indentation in juxtaposition with and spanning at least a portion of the first indentation, said second indentation being offset in a second direction from the second edge.

3. The scalpel blade of claim 2, wherein the first direction is opposite the second direction.

4. The scalpel blade of claim 2, wherein the blade is removably attached to a handle.

5. The scalpel blade of claim 2, wherein the blade is indelibly attached to a handle.

6. The scalpel blade of claim 1, wherein the blade is removably attached to a handle.

7. The scalpel blade of claim 1, wherein the blade is indelibly attached to a handle.

8. A scalpel blade having a longitudinal axis, a first edge and a second edge coincident with the longitudinal axis, and a cutting edge located along at least a portion of the first edge, said scalpel blade comprising:
a first indentation spanning at least a portion of the second edge.

9. The scalpel blade of claim 8, wherein said scalpel blade further comprises a second indentation spanning at least a portion of the first indentation.

10. The scalpel blade of claim 9, wherein:
said first indentation is offset in a first direction from the second edge; and
said second indentation is offset in a direction opposite the first direction.

11. The scalpel blade of claim 9, wherein said first indentation is located in juxtaposition with at least a portion of the second edge.

12. The scalpel blade of claim 11, wherein said second indentation is located in juxtaposition with at least a portion of the first indentation.

13. The scalpel blade of claim 9, wherein the blade is removably attached to a handle.

14. The scalpel blade of claim 9, wherein the blade is indelibly attached to a handle.

15. A scalpel blade having a longitudinal axis, said scalpel blade comprising:
a first edge coincident with the longitudinal axis;
a second edge coincident with the longitudinal axis;
a cutting edge located along at least a portion of the first edge; and
a first indentation in juxtaposition with at least a portion of the second edge, said first indentation being offset in a first direction.

16. The scalpel blade of claim 15, said scalpel blade further comprising a second indentation in juxtaposition with at least a portion of the first indentation, said second indentation being offset in a second direction.

17. The scalpel blade of claim 16, wherein the first direction is opposite the second direction.

18. The scalpel blade of claim 16, wherein:
said first indentation spans at least a portion of the second edge; and
said second indentation spans at least a portion of said first indentation.

19. The scalpel blade of claim 16, wherein the blade is removably attached to a handle.

20. The scalpel blade of claim 16, wherein the blade is indelibly attached to a handle.

21. A cutting instrument having a longitudinal axis, said cutting instrument comprising:
a first edge coincident with the longitudinal axis;
a second edge coincident with the longitudinal axis;
a cutting edge located along at least a portion of the first edge; and
a first indentation spanning at least a portion of the cutting edge, said first indentation being offset in a first direction.

22. The cutting instrument of claim 21, said cutting instrument further comprising a second indentation spanning at least a portion of the first indentation, said second indentation being offset in a direction opposite the first direction.

23. The cutting instrument of claim 22, wherein said second indentation is located in juxtaposition with at least a portion of the first indentation.

24. The cutting instrument of claim 22, wherein the cutting instrument is attached to a handle.

25. The cutting instrument of claim 21, wherein said first indentation is located in juxtaposition with at least a portion of the second edge.

26. A method of fabricating a scalpel blade from a piece of metal, said method comprising the steps of:
stamping out a blank from the metal, the blank comprising an outline of the scalpel blade having a first edge and a second edge;
stamping out an aperture from the blank;
forming a first indentation spanning at least a portion of the second edge, said first indentation being offset in a first direction; and
creating a cutting edge located along at least a portion of the first edge.

27. The method of claim 26, said method further comprising the step of forming a second indentation spanning at least a portion of the first indentation, said second indentation being offset in a second direction.

28. The method of claim 27, wherein, in the step of forming the second indentation, the second direction is opposite the first direction.

29. The method of claim 27, wherein:
in the step of forming the first indentation, the first indentation is located in juxtaposition with at least a portion of the second edge; and
in the step of forming the second indentation, the second indentation is located in juxtaposition with at least a portion of the first indentation.

30. The method of claim 27, said method further comprising the step of attaching the fabricated scalpel blade to a handle.

31. The method of claim 26, said method further comprising the step of heat treating the blank prior to the step of creating the cutting edge.

32. The method of claim 26, said method further comprising the step of attaching the fabricated scalpel blade to a handle.
